(19) United States
(12) Patent Application Publication
(10) Pub. No.: US 2002/0127275 A1
(43) Pub. Date: Sep. 12, 2002

(54) Slow release pharmaceutical compositions and methods of preparation

(75) Inventors: Bin Bin Chang, Mountain Lakes, NJ (US); Gregory P. Kushla, Florham Park, NJ (US); Jin-Wang Lai, Edison, NJ (US); Gerald P. Polli, Valley Forge, PA (US)

Correspondence Address: LAHIVE & COCKFIELD, 28 STATE STREET, BOSTON, MA, 02109, US

(73) Assignee: BASF Corporation

(21) Appl. No.: 10/051,747

(22) Filed: Jan. 11, 2002

Related U.S. Application Data
(63) Continuation of application No. 08/881,234, filed on Jun. 24, 1997, now abandoned.
(60) Provisional application No. 60/020,508, filed on Jun. 28, 1996.

(51) Int. Cl. A61K 9/26; B29B 9/00

(52) U.S. Cl. 424/470; 264/5

(57) ABSTRACT

Provided herein are slow release pharmaceutical compositions and methods of preparing slow release pharmaceutical compositions comprising mixing an unhydrated cellulose polymer with a hydrophobic substance to form a dry mixture; adding a granulating liquid to the dry mixture to form a wet mixture; and drying the wet mixture to obtain a blend suitable for use in a slow release pharmaceutical composition. The methods may be carried-out in the absence of a melting step for the hydrophobic substance. Also provided herein is a slow release composition comprising a cellulose polymer and a hydrophobic compound wherein the composition is provided in the substantial absence of a molecular coordination complex formed between the cellulose polymer and the hydrophobic substance. The slow release compositions may comprise a cellulose polymer and a hydrophobic compound in the substantial absence of a higher aliphatic alcohol.

### FIELD OF THE INVENTION

This invention is related to the field of pharmaceutical compositions and methods of preparing pharmaceutical compositions. specifically, this invention relates to slow release pharmaceutical compositions and methods of preparing them.

### BACKGROUND OF THE INVENTION

It is known to prepare pharmaceutical slow release compositions by combining lipid or hydrophobic substances with water soluble cellulose derivatives. For example, U.S. Pat. No. 4,235,870 to Leslie describes a controlled, delayed release of a therapeutically active compound from a composition having a higher aliphatic alcohol in combination with a hydrated hydroxy-alkyl cellulose. Leslie discusses how the prior art is directed to anhydrous systems used in the preparation of pharmaceutical formulations in order to avoid deleterious effects associated with aqueous systems. Leslie states that it was unexpectedly found that hydrated materials may be used to formulate slow release compositions.

Leslie also describes a method of preparing a slow-release pharmaceutical preparation comprising adding hydrated hydroxy-alkyl cellulose to molten cetyl alcohol (or to granules formed from a molten aliphatic alcohol). The examples describe that the hydroxyalkyl cellulose is hydrated by adding it to water to form a paste before it is added to the molten alcohol.

Similarly, U.S. Pat. No. 4,366,310 to Leslie describes a controlled release pharmaceutical composition comprising a coordination complex of a solvated cellulose polymer and a solid higher aliphatic alcohol. Leslie ′310 also describes compositions made by adding a solvated cellulose polymer to a melt of solid aliphatic alcohol. Leslie ′310 also describes that the cellulose is hydrated prior to adding it to a melt of the alcohol by adding the cellulose to water until a paste is formed.

U.S. Pat. 4,834,984 to Goldie, et al. describes a controlled release oral dosage form which includes dihydrocodeine, a hydrophilic polymer such as a cellulose ether, a digestible long hydrocarbon such as a higher aliphatic alcohol, and a polyalkylene glycol. The examples in Goldie et al describe a method of preparing such dosage forms wherein granules containing a hydroxyalkyl cellulose were added to a molten higher aliphatic alcohol.

U.S. Pat. 4,862,598 to Oshlack describes a controlled slow release composition comprising a combination of a higher aliphatic alcohol and an acrylic resin. The examples describe a method of preparing pharmaceutical compositions wherein a granular mass containing an acrylic resin was mixed with a melted higher aliphatic alcohol such as cetostearyl alcohol. Sustained release compositions comprising highly soluble pharmaceutical agents in a pharmaceutical carrier comprising a hydrophilic polymer dispersed in a hydrophobic matrix is described in U.S. Pat. No. 5,484,608 to Rudnic et al. The hydrophilic polymers described as being used include hydroxyalkylmethyl cellulose and acrylic acid polymers. The hydrophobic matrix is described as including parafin, alcohols such as cetearyl, waxes and behenic acid. Drugs that are described as being contemplated for use include codeine sulfate and meperidine hydrochloride.

Although these formulations are useful as sustained release compositions, there are known drawbacks to the above-described methods and compositions. One drawback is that the higher aliphatic alcohols must be melted prior to being mixed with the cellulose polymer which results in energy consumption, messy clean-up and the need to use special equipment such as water-jacketed tanks.

Another drawback is that the cellulose polymer must be hydrated prior to its being mixed with the higher aliphatic alcohol. In many cases a paste is formed as a result of hydrating the cellulose polymer. This results in extra processing steps and longer drying times. It also creates a messy material that is difficult to handle.

Accordingly, it is an object of the present invention to provide a controlled slow release pharmaceutical formulation that is prepared without the need to hydrate a cellulose polymer prior to its mixture with a hydrophobic substance. It is another object of the present invention to provide a method of preparing a controlled slow release pharmaceutical formulation without the need to melt a hydrophobic substance prior to its mixture with a cellulose polymer. Other objects and advantages of the present invention will be readily apparent from the description of the invention as set out in the specification and claims below.

### SUMMARY OF THE INVENTION

Slow release dosage forms are formulated to release active ingredients over an extended period of time. In an extended release formulation, peak plasma levels of active drug generally are attained at least two hours after administration rather than the usual one hour (or less) peak plasma level attained with immediate release dosage forms. In-vitro dissolution testing is a commonly used screening tool used in developing sustained release dosage forms. For example, a dosage form displaying in-vitro drug release of 90% over three hours might be useful as a sustained release product.

It has been unexpectedly found that a pharmaceutical slow release composition can be prepared by combining an unhydrated cellulose polymer with a hydrophobic compound. It has also been unexpectedly found that a pharmaceutical controlled slow release composition can be prepared by combining a cellulose polymer with an unmelted hydrophobic compound. Thus, a controlled slow release composition can be attained having comparable controlled release properties as prior art compositions by making the compositions in the absence of a melting step for the hydrophobic compound and/or in the absence of a hydration step for the cellulose polymer prior to mixing the cellulose polymer and the hydrophobic compound.

Accordingly, provided herein is a method of preparing a slow release pharmaceutical composition comprising mixing an unhydrated cellulose polymer with a hydrophobic substance to form a dry mixture; adding a granulating liquid to the dry mixture to form a wet mixture; and drying the wet mixture to obtain a blend suitable for use in a slow release pharmaceutical composition. Preferably, the method is carried-out in the absence of a melting step for the hydrophobic substance. Most preferably, the method is carried out in the absence of a hydrating step for the hydrophobic component of the compositions.

Also provided herein is a slow release composition comprising a cellulose polymer and a hydrophobic compound wherein the composition is provided in the substantial absence of a molecular coordination complex formed between the cellulose polymer and the hydrophobic substance. Further provided herein is a slow release pharmaceutical formulation comprising a cellulose polymer and a hydrophobic compound wherein the specific electrical conductivity of the composition in water, prior to salvation, is about the same as the sum of the separately measured specific electrical conductivities of unhydrated hydroxyalkyl cellulose polymer in water and unmelted hydrophobic compound in water. In a preferred formulation the electrical conductivity of the composition in water, prior to salvation, is nil.

The controlled slow release compositions may also comprise a cellulose polymer and a hydrophobic compound wherein the compositions are provided in the substantial absence of a higher aliphatic alcohol.

### DETAILED DESCRIPTION OF THE INVENTION

In a preferred embodiment of the invention slow release tablets are formed by mixing a hydrophobic compound with an unhydrated cellulose polymer to form a dry mixture. The mixing of the unhydrated polymer with the hydrophobic material is performed prior to the addition of a granulating liquid. Preferably, an active pharmaceutical agent is mixed with the cellulose polymer and the hydrophobic compound. Other materials or excipients may be mixed into the composition during the formation of the dry mixture. These materials may comprise fillers, diluents, lubricants, binders, granulating aids, colourants, flavourants and glidants. In a preferred embodiment of the present invention, the cellulose polymer is mixed with a hydrophobic compound, dye, and lactose.

Hydrophobic compounds suitable for use include “waxy” organic compounds that have low solubility in water and a melting point greater than room temperature. Examples of hydrophobic compounds that may be used in the present invention include: compounds selected from the group consisting of higher aliphatic acids, both saturated and unsaturated and having a carbon chain length of greater than eight; higher aliphatic esters; hydrocarbons which are solid at room temperature (e.g. wax); long chain carboxylic acids; and higher aliphatic alcohols. The preferred higher aliphatic alcohols are those selected from the group consisting of aliphatic alcohols of 8-18 carbon atoms. The most preferred aliphatic alcohols for use in the invention are lauryl alcohol, myristyl alcohol, cetyl alcohol, stearyl alcohol, and cetostearyl alcohol. Other preferred hydrophobic compounds include aliphatic acids. Preferred acids are those selected from the group consisting of palmitic, myristic, lauric, capric, stearic and behenic acid. Sodium stearyl fumarate may also be used.

The amount of the hydrophobic compound may be provided in a amount, by total weight of the composition, from about 10% to about 50%, preferably from about 10% to about 35%, most preferably from about 14% to about 28%.

The preferred cellulose polymers for use in the present invention are hydroxyalkyl cellulose polymers and hydroxyakyl methylcellulose polymers. The most preferred being hydroxymethyl, hydroxyethyl and hydroxypropyl cellulose. The preferred amount of the cellulose polymer is provided in an amount by weight of the total weight of the composition of less than 15% most preferably less than or equal to 7.5%. The ratio, by weight, of the cellulose polymer to hydrophobic compound is preferably from 1:1 to about 1:5, most preferably at about 1:3.

Preferred pharmaceutical active agents for use in the present invention include narcotic analgesics, and their salts, such as morphine, hydromorphone, diacetyl morphine, dihydromorphone, dihydrohydroxymorphinone, hydrocodone, levorphanol, methadone, pethidine, fentanyl, codeine, dihydrocodeinone, dihydrocodeine, dihydrohydroxycodeinone, propoxyphene, buprenorphine, pentazocine, nalbuphin, and butorphanol. The most preferred narcotic analgesic are those that are soluble in water but are not very soluble or freely soluble.

Other active agents suitable for use in the present invention include such drugs used for the treatment of myocardial ischemia, in particular, Ca²⁺ channel blockers such as those represented by the following classes of compounds: phenylalkylamines, dihydropyridines, benzothiazepines, diphenylpiperazines and diarylaminopropylamines. Specific compounds for use in the present invention include verapamil, diltiazem, nicardipine, nifedipine, isradipine, amlodipine, felodipine, nimodipine and bepridil. The active agents are provided in an effective amount.

The dry mixture is brought into contact with a granulating liquid capable of solvating the polymer. Such granulating liquids include water, alcohols, and ketones. Preferred liquids are methanol, ethanol, propanol and acetone. Preferably, the granulating liquid is added to the dry mixture at a constant rate until a free flowing mass is obtained. In a preferred embodiment of the invention, the cellulose must be mixed with the hydrophobic substance prior to addition of the granulating liquid. The most preferred granulating liquid is water which can be added at a temperature greater than 0° C., most preferably at room temperature. The free-flowing mass is dried to form a dried granular mixture.

Other conventional pharmaceutical materials may be added at this time such as talc and magnesium stearate. The resulting material is suitable for use in a controlled slow release pharmaceutical composition capable of being compressed into tablets or loaded into capsules. The electrical conductivities of the composition are readily determined by methods known in the art such as those described in Leslie ′310.

The following examples are illustrative only and are not meant to limit the invention in any manner.

### EXAMPLE 1

(morphine sulfate compositions containing hydroxyethyl cellulose and is cetostearyl alcohol)

30 mg morphine sulfate tablets were prepared so that the each tablet contained one part by weight of hydroxyethyl cellulose to three parts by weight of cetostearyl alcohol with the individual components as follows:

20.0% (30.0 mg) morphine sulfate pentahydrate [Mallinckrodt]

46.6% (70.0 mg) lactose monohydrate [Sheffield]

21.0% (31.5 mg) cetostearyl alcohol [Spectrum]

7.0% (10.5 mg) hydroxyethyl cellulose polymer (three different grades of material were used, NATROSOL® 250 HX, NATROSOL® 250 HHX and NATROSOL® 250 [Aqualon].

3.3% (5.0 mg) talc [Spectrum].

2.0% (3.0 mg) magnesium stearate [Mallinckrodt].

The hydroxyethyl cellulose, lactose, morphine sulfate, and cetostearyl alcohol were introduced into a Hobart mixer and mixed until a uniform mixture of fine powder was obtained. The cellulose was not mixed with water prior to being mixed with the cetostearyl alcohol. The alcohol was not melted. Deionized water was added to the mixture at a constant rate while mixing until a free-flowing sticky wet mass was obtained. The wet mass was granulated by passing the mass through a standard number 16 screen and dried at 45° C. in an oven. The dried granulation was passed though a 18 to 20 mesh sieve. The Talc and magnesium stearate were mixed into the dried granulation and the resulting blend was compressed into tablets at six, eight and/or ten kilonewtons (kN).

Dissolution testing was performed on the tablets using USP Apparatus I, pH 4.5 phosphate buffer, at 50 rpm, 37° C. These tablet were compared to 30 mg MS CONTIN® morphine sulfate tablets. Samples were collected generally at 1, 2, 3, 4, 5, and 6 hours.

The results (%) for the tablets prepared with NATROSOL® 250 HHX hydroxyethyl cellulose were as follows for the 1, 2, 3, 4, 5 and 6 hours samples (6, 8 and 10 kN tablet compression respectively): (37, 35, 36), (60, 62, 57), (71, 71, 67), (81, 80, 79), (91, 86, 96), and (96, 91, 94).

The results (%) for the tablets prepared with NATROSOL® 250 HX hydroxyethyl cellulose were as follows for the 1, 2, 3, 4, 5 and 6 hour samples (6, 8, and 10 kN tablet compression): (36, 36, 36), (53, 55, 53), (67, 68, 67), (80, 83, 81), (87, 90, 87), and (96, 96, 98).

The results for the tablets prepared with the NATROSOL® 250 L hydroxyethyl cellulose were as follows for the 1, 2, 3, 4, 5 and 6 hour samples (6, 8, and 10 kN respectively): (38, 42, 40), (61, 65, 63), (66, 65, 70), (73, 70, 70). (88, 85, 85) and (91, 91, 94).

The results (%) for the MS CONTIN® morphine sulfate tablets at 1, 2, 3, 4, 5 and 6 were: 38%, 50%, 72%, 75%, 83% and 86% respectively.

Hence, the results for all compositions ranged from 35 to 42% at one hour, 53 to 65% at two hours, 65 to 71% at three hours, 70 to 83% at four hours, 85 to 96% at five hours and 91 to 94% at six hours.

### EXAMPLE 2

(morphine sulfate compositions containing hydroxyethyl cellulose and stearic acid)

Tablets were prepared according to Example 1 except that stearic acid (USP grade, Spectrum Chemical Manufacturing Corporation) was used as the hydrophobic substance. Tablets were prepared using NATROSOL® hydroxyethyl cellulose, HHX, HX and L grades.

The results (%) for the NATROSOL® 250 HHX tablets were as follows for the 1, 2, 3, 4, 5 and 6 hours samples (6, 8 and 10 kN compression): 21, 38, 53, 64, 70, and 80.

The results (%) for the NATROSOL® 250 HX tablets were as follows for the 1, 2, 3, 4, 5 and 6 hours samples (6, 8 and 10 kN respectively): (47, 39, 41), (74, 67, 70), (81, 76, 84), (88, 80, 89), (96, 91, 100), and (95, 92, 100).

The results (%) for the NATROSOL® 250 L tablets were as follows for the 1, 2, 3, 4, 5 and 6 hours samples (6, 8, and 10 kN respectively): (65, 65, 25 59), (82, 84, 77), (108, 102, 96), (105, 109, 104), (109, 107, 103) and (110, 109,106).

The results for all compositions ranged from 21 to 65% at one hour, 38 to 84% at two hours, 53 to 108% at three hours, 64 to 109% at four hours, 70 to 109% at five hours, and 80 to 110% at six hours

### EXAMPLE 3

(morphine sulfate compositions containing hydroxyethyl cellulose and glyceryl behenate)

Tablets were prepared according to Example 1 except that glyceryl behenate (COMPRITOL® 880 ATO glyceryl behenate, NF, Gattefosse) was to used as the hydrophobic substance. Tablets were prepared using NATROSOL® hydroxyethyl cellulose, HHX, HX and L grades.

The results (%) for the NATROSOL® 250 HHX hydroxyethyl cellulose tablets were as follows for the 1, 2, 3, 4, 5 and 6 hours samples (6 kN): 52, 64, 78, 89, 99, and 101.

The results (%) for the NATROSOL® 250 HX hydroxyethyl cellulose tablets were as follows for the 1, 2, 3, 4, 5 and 6 hours samples (6, 8, and 10 respectively): (29, 26, 21), (56, 56, 53), (70, 68, 64), (82, 84, 82), (90, 88, 85), and (92, 89, 88).

The results (%) for the NATROSOL® 250 L hydroxyethyl cellulose tablets were as follows for the 1, 2, 3, 4, 5 and 6 hours samples (6, 8, and 10 kN compression respectively): (54, 49 58), (68, 60, 62), (82, 93, 87), (90, 85, 89), (96, 90, 95), and (102, 100, 103).

The results for all compositions ranged from 29 to 58% at one hour, 53 to 68% at two hours, 64 to 93% at three hours, 82 to 90% at four hours, 85 to 99% at five hours, and 88 to 103% at five hours.

### EXAMPLE 4

(morphine sulfate compositions containing hydroxyethyl cellulose and sodium stearyl fumarate)

Tablets were prepared according to the method of Example 1 except that sodium stearyl fumarate (NF, PRUV) was used as the hydrophobic substance. Tablets were prepared using NATROSOL® 250 hydroxyethyl cellulose, HHX and HX grades. Dissolution testing was performed according to the method as recited in Example 1. Samples were collected at 1, 2, 4 and 6 hours.

The results (%) for the NATROSOL® 250 HHX hydroxyethyl cellulose tablets were as follows for the 1, 2, 4, and 6 hours samples: 30, 46, 67 and 83.

The results (%) for the NATROSOL® 250 HX hydroxyethyl cellulose tablets were as follows for the 1, 2, 4, and 6 hours samples: 29, 44, 64 and 79.

MS CONTIN® morphine sulfate tablets was also tested as a control using the same dissolution parameters as in Example 1 and samples were collected at 1, 2, 3, 4, 5, and 6, hours with the results (%) as follows: 38, 50, 72, 75, 83, and 86.

The results for all compositions ranged from 29 to 30% at one hour, 64 to 66% at two hours, 64 to 67% at four hours, and 79 to 83% at six hours.

### EXAMPLE 5

(verapamil hydrochloride compositions containing hydroxyethyl cellulose and cetostearyl alcohol)

Compressed tablets were prepared as in Example 1 except that the active ingredient was verapamil instead of morphine sulfate. Further variations are that only compression forces at 6 and 12 kN were prepared for the NATROSOL® 250 HHX and HX and compression force at 5 and 10 for the NATROSOL® 250 L. Samples were collected generally at 1, 2, 3, 4, and 6 hours.

The results (%) for the NATROSOL® 250 HHX hydroxyethyl cellulose tablets were as follows for the 1, 2, 3, 4, and 6 hours samples (6 and 10 kN compression respectively): (24,21), (45, 42), (56, 53), (62, 63), and (76, 74).

The results (%) for the NATROSOL® 250 HX hydroxyethyl cellulose tablets were as follows for the 1, 2, 3, 4, and 6 hours samples (6 and 12 kN compression respectively): (15, 20), (30, 33), (49, 48), (62, 62) and (75, 76).

The results (%) for the NATROSOL® L hydroxyethyl cellulose tablets were as follows for the 1, 2, 3, 4, and 6 hours samples (5 and 10 kN compression respectively): (26, 27), (40, 42), (53, 51), (62, 60) and (71, 70).

The results for all compositions ranged from 15 to 27% at one hour, 30 to 45% at two hours 48 to 56% at three hours, 60 to 63% for at four hours, and 70 to 76% at six hours

### EXAMPLE 6

(verapamil compositions containing hydroxyethyl cellulose and stearic acid)

Tablets were prepared according to Example 4 except that stearic acid (Spectrum Chemical Manufacturing Corporation) was used as the hydrophobic substance. Compression force of 6 and 10 kN were used for the NATROSOL® 250 HHX and HX grades.

The results (%) for the NATROSOL® 250 HHX hydroxyethyl cellulose tablets were as follows for the 1, 2, 3, 4, and 6 hours samples (6 and 10 kN compression respectively): (24, 27), (40, 41), (61, 62), (70, 71), and (79, 80).

The results (%) for the NATROSOL® 250 HX hydroxyethyl cellulose tablets were as follows for the 1, 2, 3, 4, and 6 hours samples (6 and 10 kN compression respectively): (19, 22), (40, 41), (57, 57), (65, 65), and (73, 73).

The results (%) for the NATROSOL® 250 L hydroxyethyl cellulose tablets were as follows for the 1, 2, 3, 4, and 6 hours samples (4.5 and 11 kN compression respectively): (29, 29), (54, 57), (72, 69), (77, 76) and (80, 80

The results for all compositions ranged from 19 to 29% at one hour, 40 to 57% at two hours, 57 to 72% at three hours 65 to 77% at four hours, and 73 to 80% at six hours.

### EXAMPLE 7

(verapamil hydrochloride compositions containing hydroxyethyl cellulose and glyceryl behenate)

Tablets were prepared according to Example 5 except that glyceryl behenate (COMPRITOL® 880 ATO, glyceryl behenate, NF, Gattefosse) was used as the hydrophobic substance. Compression force of 6 and 12 kN were used for the tablets made with NATROSOL® 250 HHX hydroxyethyl cellulose, 6 and 10 kN were used for the tablets made with NATROSOL® 250 HX hydroxyethyl cellulose, and 5 and 10 kN were used for the tablets made with the NATROSOL® 250 L hydroxymethylcellulose.

The results (%) for the NATROSOL® 250 HHX hydroxyethyl cellulose tablets were as follows for the 1, 2, 3, 4, and 6 hours samples (6 and 12 kN compression respectively): (29, 29), (46, 47), (58, 59), (68, 70), and (79, 81).

The results (%) for the NATROSOL® 250 HX hydroxyethyl cellulose tablets were as follows for the 1, 2, 3, 4, and 6 hours samples (6 and 10 kN compression respectively): (13, 15), (36, 38), (54, 55), (64, 65), and (76, 78).

The results (%) for the NATROSOL® 250 L hydroxyethyl cellulose tablets were as follows for the 1, 2, 3, 4, and 6 hours samples (5 and 10 kN compression respectively): (24, 24), (50, 49), (63, 62), (84, 83) and (81, 81).

The results for all compositions ranged from 13 to 29% at one hour, 36 to 50% at two hours, 54 to 63% at three hours 64 to 84% at four hours, and 76 to 81% at six hours.

The methods and compositions described above provide formulations suitable for use in solid oral dosage forms used for the slow release of an effective amount of a pharmacologically active compound contained within the compositions. The compositions provided herein release active agents within the gastrointestinal track over a sustained period of time and preferably at set predetermined rates. These compositions allow for the delivery of active agents, such as morphine, to provide for effective sustained relief of pain in a mammal, preferably a human patient.

The invention has been described with reference to various specific embodiments. However, many variations and modifications may be made while remaining within the scope and spirit of the invention.

We claim:

1. A method of preparing a slow release pharmaceutical composition comprising:
(a) mixing a cellulose polymer with a hydrophobic substance to form a dry mixture;
(b) mixing a granulating liquid with the dry mixture to form a wet mixture;
(c) drying the wet mixture to obtain a blend suitable for use in a slow release pharmaceutical composition; wherein said method is carried-out in the absence of a hydration step for the cellulose polymer prior to mixing the granulating liquid with the dry mixture.

2. The method as recited in claim 1 wherein said method is carried out in the absence of a melting step for the hydrophobic substance.

3. The method of claim 1 wherein the hydrophobic substance comprises a compound selected from the group consisting of higher aliphatic acids, higher aliphatic esters, hydrocarbons which are solid at room temperature, and higher aliphatic alcohols.

4. The method of claim 2 wherein the hydrophobic substance comprises a compound selected from the group consisting of higher aliphatic acids, higher aliphatic esters, hydrocarbons which are solid at room temperature, and higher aliphatic alcohols.

5. The method of claim 1 wherein the hydrophobic substance comprises a higher aliphatic alcohol selected from the group consisting of aliphatic alcohols of 8-18 carbon atoms.

6. The method of claim 2 wherein the hydrophobic substance comprises a higher aliphatic alcohol selected from the group consisting of aliphatic alcohols of 8-18 carbon atoms.

7. The method of claim 5 wherein the higher aliphatic alcohol is lauryl alcohol, myristyl alcohol, cetyl alcohol, stearyl alcohol, or cetostearyl alcohol.

8. The method of claim 6 wherein the higher aliphatic alcohol is lauryl alcohol, myristyl alcohol, cetyl alcohol, stearyl alcohol, or cetostearyl alcohol.

9. The method of claim 1 wherein the hydroxyalkyl cellulose is selected from the group consisting of hydroxymethyl cellulose, hydroxyethyl cellulose, or hydroxy propyl cellulose.

10. The method of claim 2 wherein the hydroxyalkyl cellulose is selected from the group consisting of hydroxymethyl cellulose, hydroxyethyl cellulose, or hydroxypropyl cellulose.

11. The method of claim 3 wherein the hydroxyalkyl cellulose is selected from the group consisting of hydroxymethyl cellulose, hydroxyethyl cellulose, or hydroxypropyl cellulose.

12. The method of claim 4 wherein the hydroxyalkyl cellulose is selected from the group consisting of hydroxymethyl cellulose, hydroxyethyl cellulose, or hydroxypropyl cellulose.

13. The method of claim 5 wherein the hydroxyalkyl cellulose is selected from the group consisting of hydroxymethyl cellulose, hydroxyethyl cellulose, or hydroxypropyl cellulose.

14. The method of claim 6 wherein the hydroxyalkyl cellulose is selected from the group consisting of hydroxymethyl cellulose, hydroxyethyl cellulose, or hydroxypropyl cellulose.

15. The method of claim 7 wherein the hydroxyalkyl cellulose is selected from the group consisting of hydroxymethyl cellulose, hydroxyethyl cellulose, or hydroxypropyl cellulose.
